(19) United States
(12) Patent Application Publication
(10) Pub. No.: US 2023/0157752 A1
(43) Pub. Date: May 25, 2023

(54) Stimulation Device

(71) Applicant: Filip Sedic, Stockholm (SE)

(72) Inventor: Filip Sedic, Stockholm (SE)

(21) Appl. No.: 17/985,337

(22) Filed: Nov. 11, 2022

Related U.S. Application Data
(60) Provisional application No. 63/282,719, filed on Nov. 24, 2021.

(51) Int. Cl. A61B 18/18; A61H 23/02

(52) U.S. Cl. CPC A61B 18/18; A61H 23/02; A61H 2201/10; A61B 2018/1807; A61B 2018/00476

(57) ABSTRACT

An example treatment device includes a main body having a first end, a second end, an intermediate portion, a light source, a capacitor, a motor, and a controller. The treatment device may be configured to provide a user with one or more of intense pulsed light treatment, hair removal, and/or vibrational stimulation.

### FIELD

The disclosure relates generally to the field of treatment devices and methods of use. More particularly, the disclosure relates to treatment devices suitable for use on the human face and body and various methods of using the same.

### BACKGROUND

Intense pulsed light (hereinafter, “IPL”) devices are devices which utilize various wavelengths of light generated as output via a device to provide particular benefits. One known function of IPL devices involves the destruction of hair follicles beneath a user's skin to prevent unwanted hair growth. In such instances, IPL devices direct various wavelengths of light at one or more follicles, which destroy hair follicles by selectively heating and cooling said hair follicles while avoiding damaging skin or tissues near the area to be treated. Photothermolytic destruction of the hair follicles occurs in such instances between the darker colored follicles and/or melanin residing in the hair follicles, which absorbs light and heat, allowing the same to be destroyed. IPL devices may also provide additional, non-hair removal related benefits, including aesthetic photo rejuvenation and treatment of skin disorders.

Several devices exist which may provide IPL treatments to a user. Such devices, however, typically take a significant amount of time to charge, as they include only one capacitor. Additionally, such devices only include one light source, which ultimately requires that said light source be replaced frequently. Finally, such devices do not provide vibrational or electronic muscle stimulation.

A need exists, therefore, for improved treatment devices and methods of use that provide improved IPL treatments to a user.

### BRIEF SUMMARY OF SELECTED EXAMPLES

Various example treatment devices and methods of use are described.

An example treatment device comprises a main body having a first end, a second end substantially opposite the first end, an intermediate portion extending from the first end to the second end, a head disposed adjacent the first end, a first light source disposed within the head and configured to emit light adjacent the head, a motor disposed within the main body, the motor configured to produce pulsations of said treatment device, a first capacitor disposed within the main body, the capacitor configured to supply power to the first light source, a second capacitor disposed within the main body, the capacitor configured to supply power to the second light source; and a set of user control disposed on the intermediate portion, the set of user controls configured to operate the first and second light sources.

Additional understanding of claimed devices and methods may be obtained by reviewing the detailed description of selected examples, below, with reference to the appended drawings.

### DESCRIPTION OF FIGURES

FIG. 1 is a perspective view of a first example treatment device.

FIG. 2 is an end view of the light treatment device illustrated in FIG. 1.

FIG. 3 is a side view of the treatment device illustrated in FIG. 1.

FIG. 4 is a sectional view of the treatment device illustrated in FIG. 3, taken along line 4-4.

FIG. 5 is another side view of the treatment device illustrated in FIG. 1.

FIG. 6 is another end view of the treatment device illustrated in FIG. 1.

FIG. 7 is a top view of the treatment device illustrated in FIG. 1.

FIG. 8 is a bottom view of the treatment device illustrated in FIG. 1.

FIG. 9 is a diagram illustrating components of a networked treatment device in an example embodiment.

FIG. 10 is a perspective view of a second example treatment device.

FIG. 11 is an end view of the treatment device illustrated in FIG. 10.

FIG. 12 is a side view of the treatment device illustrated in FIG. 10.

FIG. 13 is another side view of the treatment device illustrated in FIG. 10.

FIG. 14 is another end view of the treatment device illustrated in FIG. 10.

FIG. 15 is a top view of the treatment device illustrated in FIG. 10.

FIG. 16 is a bottom view of the treatment device illustrated in FIG. 10.

FIG. 17 is a perspective view of a third example treatment device.

FIG. 18 is an end view of the treatment device illustrated in FIG. 17.

FIG. 19 is a side view of the treatment device illustrated in FIG. 17.

FIG. 20 is a sectional view of the treatment device illustrated in FIG. 17, taken along line 20-20.

FIG. 21 is another side view of the treatment device illustrated in FIG. 17.

FIG. 21A is a sectional view of an alternative treatment device.

FIG. 22 is another end view of the treatment device illustrated in FIG. 17.

FIG. 23 is a top view of the treatment device illustrated in FIG. 17.

FIG. 24 is a bottom view of the treatment device illustrated in FIG. 17.

FIG. 25 is a perspective view of a fourth example treatment device.

FIG. 26 is an end view of the treatment device illustrated in FIG. 25.

FIG. 27 is a side view of the treatment device illustrated in FIG. 25.

FIG. 28 is a sectional view of the treatment device illustrated in FIG. 25, taken along line 28-28.

FIG. 29 is another side view of the treatment device illustrated in FIG. 25.

FIG. 29A is a sectional view of an alternative treatment device.

FIG. 30 is another end view of the treatment device illustrated in FIG. 25.

FIG. 31 is a top view of the treatment device illustrated in FIG. 25.

FIG. 32 is a bottom view of the treatment device illustrated in FIG. 25.

FIG. 33 is a flowchart representation of an example method of using a treatment device.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF SELECTED EXAMPLES

The following detailed description and the appended drawings describe and illustrate various treatment devices and methods of use. The description and drawings are provided to enable one skilled in the art to make and use one or more example treatment devices. They are not intended to limit the scope of the claims in any manner.

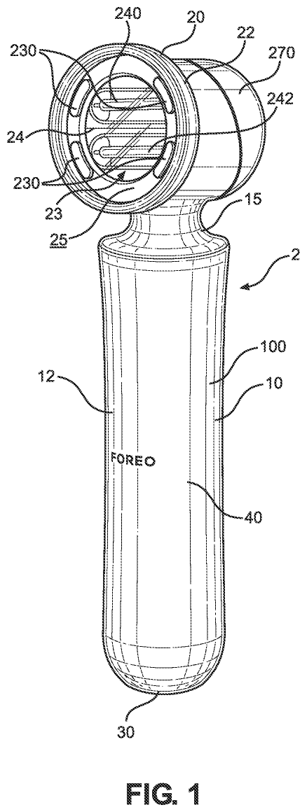

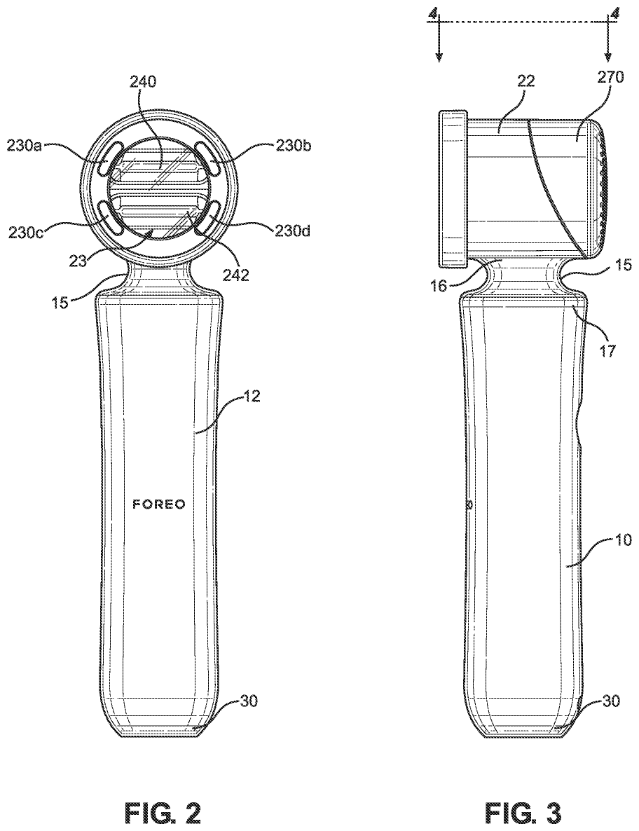

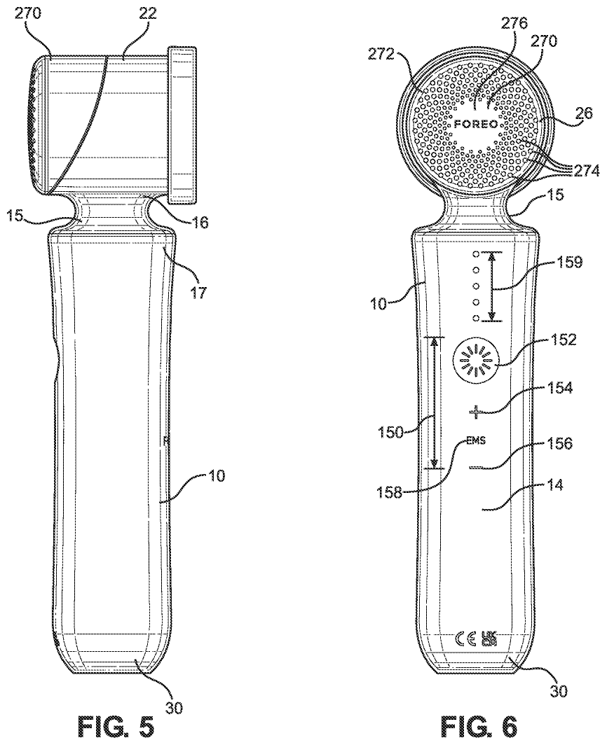

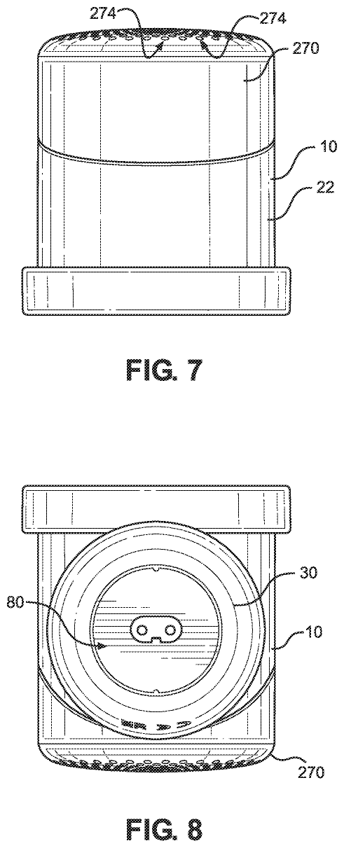

FIGS. 1, 2, 3, 4, 5, 6, 7, 8, and 9 illustrate an example treatment device 2 (hereinafter, also referred to as the “device”). The device 2 comprises a main body 10 that includes and/or houses several components (described in greater detail below).

The main body 10 includes at least a first end 20, a second end 30 substantially opposite the first end 20, and an intermediate portion 40 extending from the first end 20 to the second end 30. The first end 20 includes a head 22 (which includes a head shell 23), which may be placed in contact with or adjacent a user's skin in order to provide intense pulsed treatment (“IPL treatment”). The intermediate portion 40 and second end 30 generally provide a consistent size and shape such that a user may hold the device with one hand and easily grasp the same. The second end 30 may optionally be configured such that the device 2 can stand on a flat surface unaided, while not in use. The device 2 is configured such that the front 12 of the device 2 provides treatment to a user, while the rear 14 of the device controls the functionality of the device 2 via a set of controls (described further below). Specifically, a user may grasp the device 2 and press one or more of the controls disposed on the rear 14 of the device 2 with his or her thumb. Internal components, including a motor 200, first and second controllers 212, 210, first and second capacitors 220, 222, a set of electronic muscle stimulation (“EMS”) pins 230, and other components are housed within the main body 10 of the device 2 as well. The main body 10 includes a body shell 11, comprised of plastic like the head shell 23 is, which forms a portion of the main body 10 of the device 2. The body shell 11 generally includes most of the intermediate portion 40 and the second end 30. The body shell 11 is disposed adjacent neck 15, which connects the head shell 23 to the intermediate portion 40 and second end 30. The neck 15 has a first end 16 and second end 17; the first end 16 is adjacent the head 22, while the second end 17 is adjacent the intermediate portion 40. The neck 15 is formed such that the head 22 and the portion of the intermediate portion 40 adjacent the neck 15 are able to attach to the neck to form the device in production; however, once formed, the head 22, neck 15, and intermediate portions 40 cannot be taken apart from one another by a user. The neck 15 is comprised of a neck shell 18 surrounded by rubber that can be turned and twisted while in use (or before such use) to allow the head 22 to be placed adjacent a desired treatment area with ease. The neck 15 is substantially self-adjustable such that, when a user's force is no longer exerted on the same, it will generally return to a base position. A skilled artisan will be able to determine how best to form the neck, head, and remainder of the main body according to a particular example based on various considerations, including the desired flexibility of the device, the size and shape of the head, and the functionality of the device. In other embodiments, the main body comprises one shell. In another embodiment, the neck may not include a shell and may only include a rubber piece, which may or may not be flexible. In different embodiments, the neck shell, head shell, and body shell may comprise a single integral piece. In various embodiments, the neck may attach with the head and body shell via snap-fit mechanisms, adhesives, or other physical connection mechanisms.

The device 2 also includes a silicone exterior 100, which covers and is attached to (and is non-removable from) the head shell 23, neck 15, and body shell 11 in the current embodiment. The silicone exterior 100 is comprised of medical-grade silicone in the illustrated embodiment and extends from the second end 30 to the first end 20 (while covering the intermediate portion 40) of the device 2. It covers at least a portion of the neck 15 and head 22, as well. A skilled artisan will be able to select a suitable shape and size of the device according to a particular example based on various considerations including the desired functionality of the device and any desired potential treatment locations. In other embodiments, the main body may be curved or have any other shape. Additionally, in different embodiments the head may be detachable, permanently affixed to the main body, and have various other shapes. In another embodiment, the silicone exterior may cover all, a portion of, or none of the main body; the silicone exterior may also have perforations, designs, or be formed in other ways. It may also be permanently affixed to the shell via a physical mechanism or an adhesive; it may also be configured such that it is removable from the shell when in use. The shell may be comprised of materials other than plastic in different embodiments, as well.

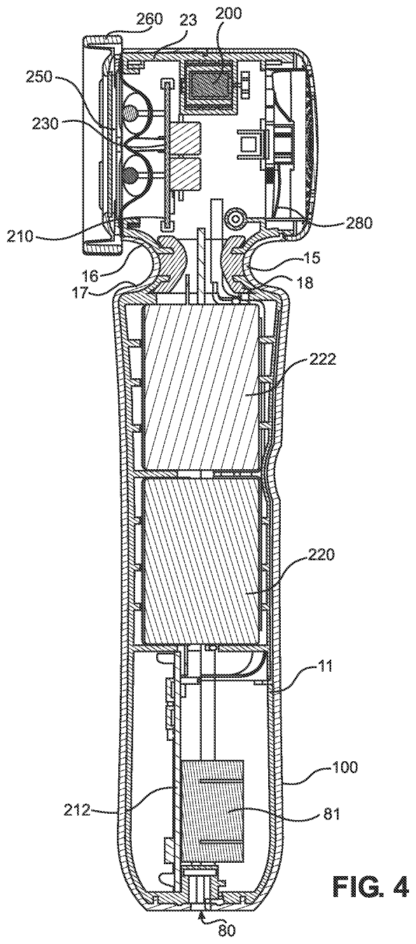

The head 22 of the main body 10 includes a first side 24 and a second side 26 substantially opposite the first side 24. The first side 24 of the head 22 is configured to be positioned in contact with or directly adjacent a user's skin in order to provide various types of treatment to a user. The head 22 houses first and second light sources 240, 242, which are operatively connected with various internal components and configured to treat the skin with light; they comprise xenon discharge lamps in this embodiment. The first and second light sources 240, 242 are substantially cylindrical in shape and are disposed within a recessed portion 23 of the head 22. This recessed portion 23 is indented relative to other portions of the first side 24 of the head 22. As shown in FIG. 4, it also houses a reflector 250 and a glass filter 260, which cooperatively work with the device's 2 other components to allow IPL treatment to be applied to a user. The glass filter 260 and first and second light sources 240, 242 are at least partially comprised of glass in the illustrated embodiment. The reflector 250 comprises stamped and coated aluminum. These components are held in place within the head 22 via physical connecting mechanisms which hold the glass filter 260, reflector 250, and first and second light sources 240, 242 in place. The first and second light sources 240, 242 are configured to be activated once the capacitors 220, 222 are sufficiently charged. The reflector 250 helps to strength and concentrate the light emitted by the first and second light sources 240, 242. The glass filter 260 helps to filter out potentially harmful types of light, such as UV light, so that the device will not harm a user. In other embodiments, the light sources may comprise any type of lamp, glass, LED light, or other mechanism of producing light that may penetrate the skin to treat hair follicles. In different embodiments, the glass filter and reflector may be comprised of any suitable material, including suitable metals, plastics, glasses, and the like. In other embodiments, the device may include one, two, three, four, or more than four light sources. The light sources may also be aligned relative to one another in any shape or format. For example, the light sources may be adjacent one another, stacked atop one another, in a triangular, square, circular, or other formation, or may be arranged without a set pattern. A skilled artisan will be able to determine a suitable arrangement for the light sources.

A portion of the second side 26 of the head 22 comprises metal in the illustrated embodiment. More specifically, the second side 26 includes a metallic portion 270 that is connected to the shell 11 and positioned substantially at the first end 20 of the device 2. The metallic portion 270 includes a surface 272 defining a set of passageways 274 extending from the surface 272 to an interior surface (not illustrated in the embodiments). These passageways 274 have substantially circular openings and vary slightly in diameter such that, generally, passageways having larger diameters are further from a center 276 of the metallic portion 270 than are passageways having smaller diameters. Additionally, the center 276 of the metallic portion 270 does not include passageways. The passageways 274 (alternatively thought of as a fluid outlet) are designed to allow warm air that is generated when the device 2 is in use to dissipate through the metallic portion's 270 passageways 274 and away from the device 2. A fan 280 is also disposed within the head 22. The fan 280 is disposed adjacent the passageways 274 and further helps to move warm air away from the device and out the second side 26. This helps to ensure that the device 2 does not overheat and become non-functional. A skilled artisan will determine how best to configure the metallic portion and passageways according to a particular example based on various considerations, including the potential heat generated by the device and size and shape of the head. In different embodiments, the head may be comprised of any suitable metal, including aluminum, titanium, an alloy, or any other suitable material; in other embodiments, the head may be comprised of plastic or any other non-metal. In different embodiments, the metallic piece and/or head may be integrally formed with the rest of the main body; in alternative embodiments, either may be attached to other portions of the main body via an adhesive or physical attachment mechanism. In other embodiments, the passageways may have any shape, diameter, and alignment; in such examples, the passageways may have triangular, square, rectangular, or other opening shapes. The metallic portion may also include 0, 1, 5, 10, or more than 10 passageways in different embodiments.

The device 2 includes first and second controllers 212, 210 disposed within the main body 10, as well. The first controller 212 and the second controller 210 in the illustrated embodiment include a printed circuit board assemblies (“PCBA”) and related circuitry; however, in other embodiments, either controller may comprise any device suitable to control the device's components. The first controller 212 is disposed within the head 22 of the device 2 and is configured to communicate with the first and second light sources 240, 242. The second controller 210 is disposed substantially adjacent the intermediate portion 40 and is involved with the charging of the first and second capacitors 220, 222. When the device 2 is charged, the first controller 212 and second controller 210 communicate with each other and the various other components of the device in order to activate the first and second light sources 240, 242; the first and second controllers 212, 210 first, however, ensure that the first and second capacitors 220, 222 are charged to such a degree that the device 2 can be properly activated. In other embodiments, either controller can communicate with any other of the device's components.

Moreover, one or both of the first and second controllers 212, 210 be operatively connected to an interface 90, allowing one or both to communicate with a second device (described in greater detail below). In the current embodiment, the second controller 210 communicates with a second device; in other embodiments, one or both of the controllers may do so.

The device 2 can also connect to an external power source via a DC jack 80 (best illustrated in FIG. 4), which couples to a battery 81 that powers the device 2 when in use. The DC jack 80 is configured such that it may operatively connect to an external power source, such as a wall plug, via an adaptor or the like. Once the device 2 is operatively connected to an external power source, it provides power to the various components of the device 2 so that they may function efficiently and properly. The DC jack 80 is securely placed within second end 30 of the main body 10. In the illustrated embodiment, the device 2 must be connected to an external power source in order to operate. A skilled artisan will be able to select a suitable mechanism of providing power to the device according to a particular example based on various considerations, including the various components housed within the device and the desired dimensions of the device. In other embodiments, the power source may comprise one, two, three, or more than three replaceable dry cell batteries. In different embodiments, the power source may comprise a lithium polymer battery or multiple such batteries. In different embodiments, the DC jack may be replaced by any suitable mechanism to provide power to the device; the device may also operate when not connected to a power source in other embodiments.

A motor 200 is also housed within the first end 20 of the main body 10. It is operatively connected to the first controller 212. The motor 200 is at least partially disposed within in a frame 202 disposed within the main body 10. The frame 202 is configured to support the motor 200 and keep the motor 200 secured at a particular location inside the main body 10, as well as provide general structural support to the treatment device 2. Specifically, the frame 202 includes an arm that defines a slot into which the motor 200 is disposed. The motor 200 is disposed within the head 22 so its vibrations may be efficiently transmitted to the user via the head 22.

One or both of the first and second controllers 212, 210 provides instructions to the motor 200 when the device 2 is in operation. A high or low-frequency motor 200 may be used to create pulsations that vibrate the treatment device in various embodiments. The motor 200 may comprise an oscillating motor. The motor 200 in this embodiment is configured to produce a range of frequencies that may provide beneficial treatment of the skin. When the treatment device 2 is in operation, and the head 22 in particular, is applied to the body, such as the face or neck, the motor 200 provides vibrations to the skin. Such vibrations have known benefits, including stimulation collagen production and muscle toning. A skilled artisan will be able to select suitable motors, frames, and vibration ranges based on various considerations, including the size and shape of the treatment device and the desired vibration strength of the device. Examples of suitable ranges for vibration frequencies includes vibration frequencies between about 80 Hertz (“Hz”) and about 200 Hz, vibration frequencies between about 100 Hz and about 180 Hz, and vibration frequencies between about 120 Hz and about 150 Hz. In another embodiment, the device may include two or more motors. In another embodiment, a motor may be stochastic. In other embodiments, the frame may be omitted. In different embodiments, the frame may not include a slot or arm, but may instead house the device entirely within its main body. In some embodiments, there may be more than one motor; in embodiments containing multiple motors, the motors may vary from one another in frequency output. In other embodiments, the motor may be controlled by the second controller. The motor also may be placed anywhere within the main body in alternative embodiments.

One or both of the first and second controllers 212, 210, optionally, also controls an interface 90 that is a component of the second controller 210. The interface 90 allows the treatment device 2 to communicate with a second device, such as a personal computer, tablet, mobile telephone, or other electronic device (not illustrated in the Figures). Using the interface 90, the treatment device 2 can send information to other devices so that the other device(s) may collect data pertaining to the use of the treatment device 2. Furthermore, the treatment device 2 may receive control signals from another device that can indicate that the treatment device 2 should turn on or off, increase or decrease speed, switch to a different vibration or lighting pattern, and/or switch to a pre-set pattern desired by the user or recommended by the other device, among other instructions. The interface 90 can be a wired or a wireless interface, such as a wireless transceiver that transmits control signals between the treatment device 2 and the second device. A skilled artisan will be able to select a suitable interface based on various considerations, including the device with which the treatment device will communicate and the size and shape of the main body. In some embodiments, the interface is a radiofrequency (“RF”) transceiver used to transmit and receive RF signals between the treatment device and other devices. One example of an RF transceiver that could be used is a low power 2.4 GHz RF transceiver. In various embodiments, the treatment device may also include antennas for transmitting and receiving signals between the treatment device and other devices. In such examples, the interface can use BLUETOOTH®, Wi-Fi, infrared, laser light, visible light, acoustic energy, or one of several other methods to transmit information wirelessly between the treatment device and another device. In another embodiment, one or both controllers can specifically communicate with another device to confirm the treatment device's authenticity.

In some embodiments, the treatment device is connected to a network via the second device. In other embodiments, the treatment device is directly connected to a wireless router or cellular phone network and may connect with the second device in any of said manners. Accordingly, the treatment device can be controlled via personal computer, tablet, mobile phone, or other suitable electronic devices a user using the personal computer, tablet, phone, or other device. An application on a mobile phone, for instance, may communicate with the treatment device in order to control the treatment device's functionality.

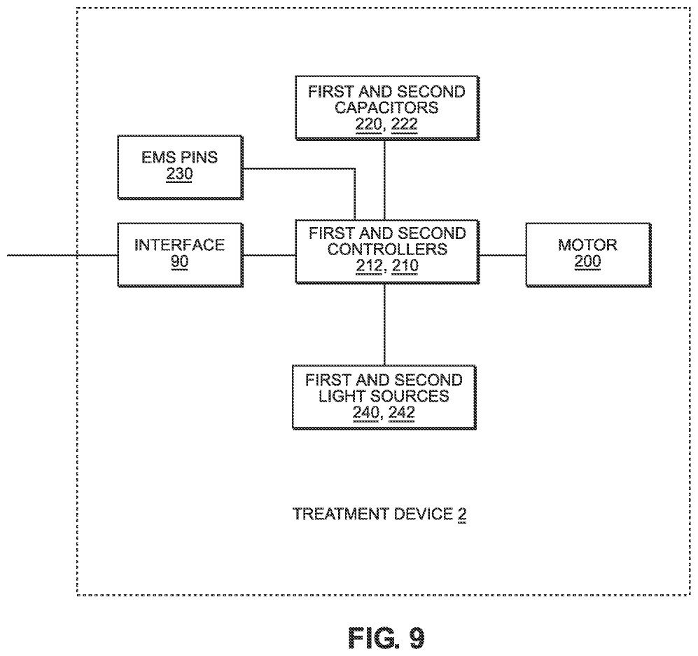

FIG. 9 is a diagram illustrating components of a networked treatment device, such as treatment device 2, in accordance with an example embodiment. In this embodiment, the treatment device includes the first and second controllers 212, 210, the motor 200, the first and second light sources 240, 242, a set of pins 230, and an interface, such as interface 90. As explained above, the treatment device can be connected to a network via a personal computer, tablet, mobile telephone, or other electronic device or can be directly connected to a wireless router or cellular phone network. Thus, the treatment device can be ,controlled by, transmit data to, and/or receive data from the personal computer, tablet, mobile telephone, or other electronic device via the aforementioned mechanisms. The interface may be wired or wireless and may include any of those described above. A skilled artisan will be able to determine how to suitably connect the treatment device with other devices according to a particular example based on various considerations, including the desirability of doing so and the devices to which connection would be beneficial. In some embodiments, the treatment device may not include an interface and, thus, may not communicate with other devices. In different embodiments, the treatment device may only transmit data to other devices; it may not receive any data and cannot be controlled via said other devices in this embodiment.

Example data that the treatment device may communicate to one or more of a personal computer, tablet, mobile telephone, or another electronic device may include the number of uses of the treatment device, the durations of the various uses of the treatment device, the user's preferred treatment device settings, and various other types of information related to the use of the device.

The main body 10 also houses first and second capacitors 220, 222, as best illustrated in FIG. 4. The first capacitor 220 is disposed closer to the second end 30 of the main body 10 than is the second capacitor 222. The first capacitor 220 is configured such that it is operatively connected to the first light source 240, while the second capacitor 222 is operatively connected to the second light source 242. The first and second capacitors 220, 222 are also operatively connected to the first and second controllers 212, 210 such that said controllers 212, 210 are able to determine when the capacitors 220, 222 are sufficiently charged (via DC jack 80) to allow for the device to be operated. The first and second controllers 212, 210 and the set of pins 230, cooperatively determine whether a user is contacting the head 22 of the device 2 such that the device 2 should become operational. More specifically, the first and second capacitors 220, 222 include capacitive sensors and related circuitry that allow for the device 2 to be programmed such that it will not activate (or “tire”) the first and second light sources 240, 242 unless a user places the appropriate portion of the head 22 of the device 2 against skin. Safety dictates that the device 2 operate this way; if it did not, the first and second light sources 240, 242 may fire into a person's eyes, which could harm that person. The first and second controllers 212, 210 and set of pins 230 are able to cooperatively sense the moisture in a user's body in order to determine whether, once a user control is utilized, it is an appropriate time for the device 2 to fire. A skilled artisan will be able to determine how best to configure the capacitors according to a particular example based on a number of factors, including the desired number of light sources of the device, how powerful the device may be, and the size and shape of the device. In one embodiment, the device may have one, three, four or more than four capacitors. In a different embodiment, the device may include one capacitor that communicates with both light sources. In another embodiment, the capacitors may be configured such that the device may fire when not in contact with a person. In an additional embodiment, the wavelength of light emitted by one or both light sources may be between about 300 nanometers (“nm”) and about 900 nm, between about 400 nm and about 700 nm, and between about 500 nm and about 600 nm. In an additional embodiment, the light sources may emit light in pulses, continuously, stochastically, or in a pattern. Capacitors may fire at the same time, on a rotating basis, partially at the same time, sequentially, or in any other pattern in various embodiments. They also may be aligned in any of a number of ways. In the illustrated embodiment, they are disposed adjacent one another, with one being disposed closer to the head of the device than the base. In other embodiments, however, the capacitors may be next to one another, staggered, or placed in various shapes (circles, triangles, squares, and the like, if there are 2 or more).

The light sources may comprise lamps, in other embodiments, and may comprise different types of light sources in different embodiments, including LED lights. In other embodiments, one or more capacitor, pin, and/or controller may determine whether a user is contacting the device such that it should activate.

The head 22 includes a set of pins 230 which protrude from a surface 25 of the head 22. As best illustrated in FIG. 1, the set of pins 60 includes substantially similarly shaped outer pins 230a, 230b, 230c, 230d (collectively referred to as the “pins 230”). The pins 230 are disposed around the first and second light sources 240, 242. The pins 230 are each arced in shape and are substantially the same in size. The pins 230 are comprised of a suitable metal or combination of metals in the illustrated embodiment. The pins 230 are attached to the head 22 in the illustrated embodiment via mechanical attachment mechanisms. The pins 230 work cooperatively with the first and second controllers 212, 210 to detect whether a user is contacting the head 22. A skilled artisan will be able to select suitable pins according to a particular example based on various considerations including the size and shape of the base, the desired functionality of the device, and any desired potential treatment locations. In other embodiments, the base may include one, two, three, four, six, or more than six pins. In different embodiments, the one or more pins may have any shape and any individual pin may be shaped the same as or differently than any other individual pin. In another embodiment, the pins may be integrally formed with the base, adhesively attached to the base, or otherwise connected to the base. In yet another embodiment, the pins may be comprised of any suitable material, including zinc or other suitable metals, which may be gold-plated (including 18 carat gold plated) or titanium plated.

The pins 230 are configured such that they each can transmit electronic muscle stimulation to a user. The pins 230 are operatively connected to the first and second controllers 212, 210 such that a user may select when and whether to utilize the pins 230 for transmission of electronic muscle stimulation while he or she uses the IPL and/or vibrational treatments of the device 2. In various other embodiments, one or more of the pins may transmit radiofrequency stimulation. In another embodiment, the electronic muscle stimulation may be emitted in pulses when the device is in use. When in use, the device 2 produces stimulation that may be applied to the skin and muscles and tissues disposed beneath the skin, which can strengthen the muscles and tissues adjacent the skin. A skilled artisan will be able to determine the number, size, shape, and configuration of the pins and will be able to suitably determine which pins should emit electronic muscle stimulation or radiofrequency stimulation according to a particular example based on various considerations, including the desired functionality of the device and the size and shape of the same. In various embodiments, the device may include one, two, three, five, six, or more than six pins. In other embodiments, the pins may be configured in any manner relative to one another and may have any size and/or shape. In an additional embodiment, the frequency of the radiofrequency stimulation emitted by the device may be between about 0.5 megahertz (“MHz”) and about 5 MHz, between about 1 MHz and about 4 MHz, and between about 1.5 MHz and about 2 MHz. In an additional embodiment, the electronic muscle stimulation emitted by the device may be between about 10 Hz and about 200 Hz, between about 25 Hz and 100 Hz, and between about 40 Hz and about 50 Hz. In various embodiments, the device may emit electronic muscle stimulation pulses; the device may emit between about 10 pulses per second (“pps”) and 200 pps, between about 50 pps and about 150 pps, and between about 100 pps and about 110 pps.

In another embodiment, each of the pins contains a mechanism (not illustrated in the Figures) that allows for the pins to alternate between transmission of electronic muscle stimulation and radiofrequency stimulation. This mechanism is a physical mechanism that communicates with the second controller in the illustrated embodiment; however, in other embodiments, it may be an electronic mechanism that communicates with one or both controllers, a software messaging system that ensures smooth alternating between the two modes, and/or any other similar mechanism.

A set of user controls 150 is disposed on the second side 14 of the treatment device 2 and is configured to allow a user to control one or more aspects of the device 2. The set of user controls 150 is operatively connected to the first and second controllers 212, 210 and, upon activation of one or more controls, the device 2 to perform one or more functions and/or actions. The set of user controls 150 comprises a first button 152 in the illustrated embodiment, which allow for a user to activate the treatment device 2 by pressing said first button 152. The second and third buttons 154, 156 are disposed adjacent the first button 152 and are configured to allow the user to increase or decrease the intensity of a particular treatment (including vibrational and IPL treatment). More specifically, the second button 154 allows a user to increase the intensity of such a treatment, while the third button 156 allows a user to decrease the same. The fourth button 158 controls the EMS output of the device 2. Specifically, when pressed, the fourth button 158 will instruct the first and second controllers 212, 210 to initiate EMS treatment via the set of pins 230. In one embodiment, the fourth button 158 may be held to produce EMS treatment; in others, the button may be pressed once to activate such treatment, and again to stop it. The set of user controls 150 is disposed such that a user can easily press it while the device 2 is in use and held by a user. Optionally, repeated activation of the first button 152 allows for a user to cycle through various modes of operation of the skin treatment device 2. In various embodiments, potential modes that may be activated by the set of user controls 150 include low frequency vibration mode, high frequency vibration mode, stochastic vibration mode, alternating frequency vibration mode, heat therapy mode, electrical current stimulation mode, and/or some combination thereof. A skilled artisan will be able to suitably place the set of user controls on the device and configure the same according to a particular example based on various considerations, including the number of desired modes. In other embodiments, the set of user control may be disposed on the front of the main body, the side(s) of the main body, or the base of the main body. In a different embodiment, the treatment device may comprise zero, one, three, or more than three user controls. Optionally, the device may also include a set of informational lights, which indicate to the user the power level that has been selected; see the informational lights 159 in FIG. 6, which include four lights in this embodiment.

In use, a user may grasp the main body 10 of the treatment device 2 in his or her hand, turn the treatment device 2 on via the set of user controls 150, and place the head 22 of the device 2 adjacent to and/or in contact with a desired treatment area. The user will then select a desired functionality by pressing one or more of the controls of the set of controls 150. The user may also select a functionality (via an app or via pressing the first button 152 a number of times) prior to placing the treatment device 2 against his or her skin. The treatment device 2 will then perform the function selected by the user. At this time, the user may increase or decrease the intensity of the function via the second and third buttons 154, 156. Subsequently, the user will be able to utilize the same function again, choose and implement a second function, and/or power off the treatment device 2.

Once a treatment has been selected by a user, the treatment device 2 will do one or more of providing electronic muscle stimulation, vibration stimulation, and/or IPL and hair removal treatments. Specifically, for example, a user may press the first button 152 in order to instruct the device to perform IPL treatment. If the device 2 is connected to a power source and the first and second capacitors 220, 222 are sufficiently charged, the first and second controllers 212, 210 will instruct the first and second capacitors 220, 222 to provide IPL treatment. Next, the light sources will heat to the user's desired preference and shall pulse light from the first and second light sources 240, 242 towards a user's skin. Such light pulsations will travel through the outer layer of the skin and, if an appropriate intensity/wavelength output is achieved, shall destroy one or more hair follicles disposed within the user's body. Similarly, a user can also control vibrational and EMS output from the head 22 of the device 2 as well.

The treatment device 2 may perform these treatments in any order, for any duration, and through contact with or placement near the skin via various portions of the device and as described above. One or more treatments may be emitted at the same time. A skilled artisan will be able to determine how best to configure the device to perform such functions.

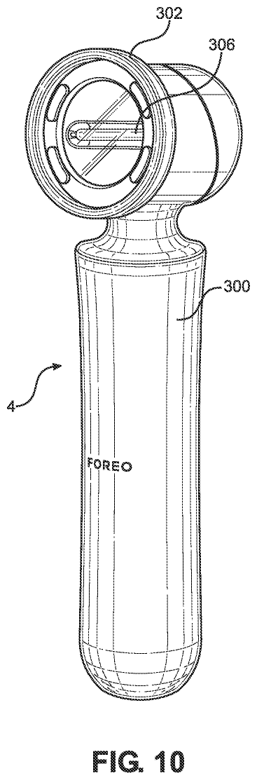

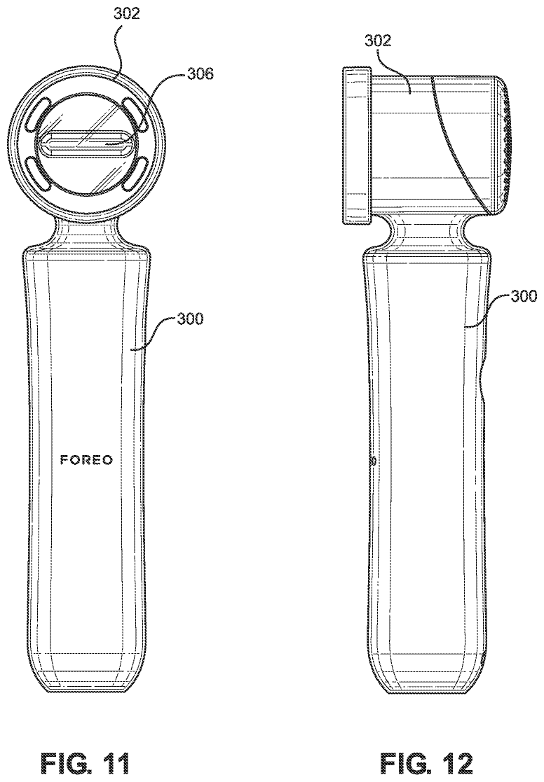

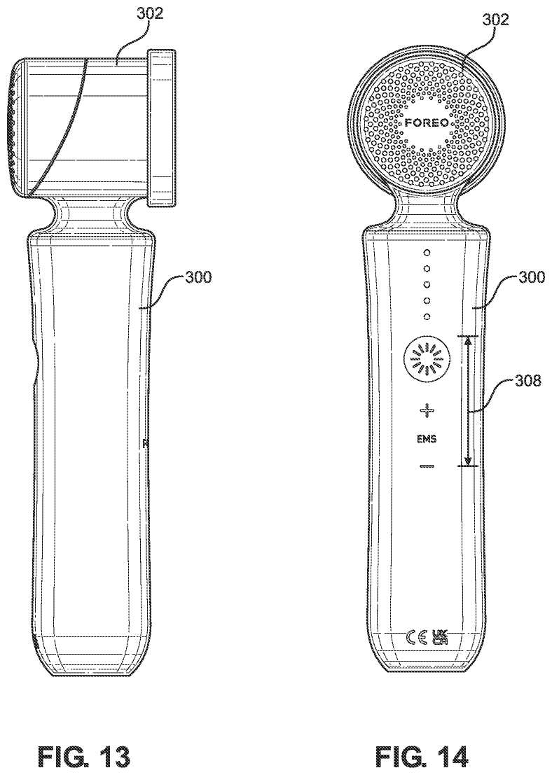

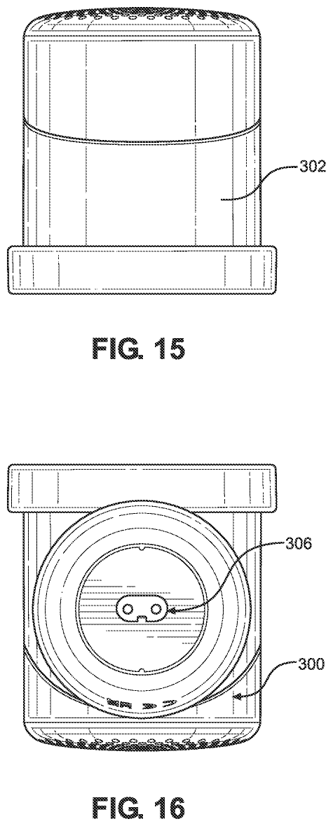

FIGS. 10, 11, 12, 13, 14, 15, and 16 illustrate another example treatment device 4. The treatment device 4 is similar to the treatment device 2 illustrated in FIGS. 1 through 9, except as described below. Thus, the device 4 comprises at least a main body 300, a head 302, internal components (including a motor and first and second controllers), a DC jack 304, a first light source 306, and a set of user controls 308.

The treatment device 4 is substantially identical to the treatment device 2 described above. However, this treatment device 4 includes only one light source 306, rather than two. It also includes only one capacitor (not illustrated in the Figures), as compared to treatment device 2 which has two capacitors.

A skilled artisan will be able to determine how many light sources and capacitors to include according to a particular embodiment based on various considerations, including the desired strength of the device and how frequently he or she would prefer to replace the light sources. In other embodiments, the device may have one, three, four, or more than four light sources; in different embodiments, the device may have zero, three, four, or more than four capacitors.

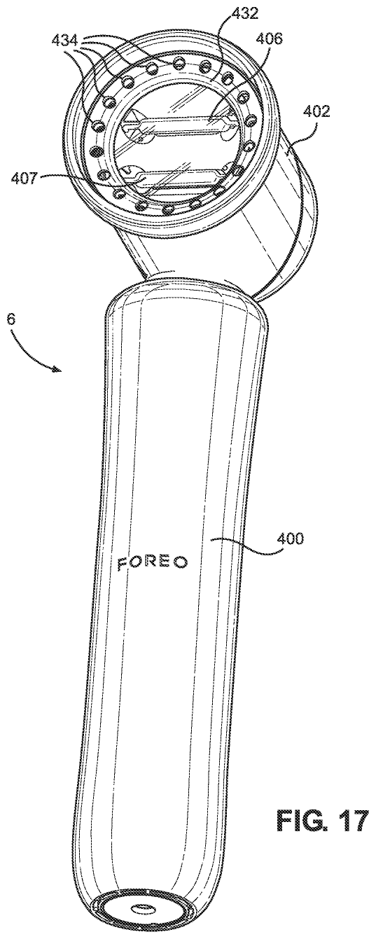

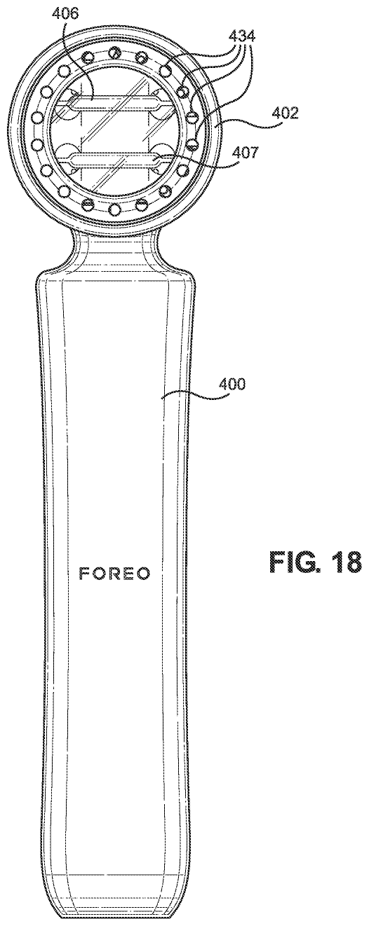

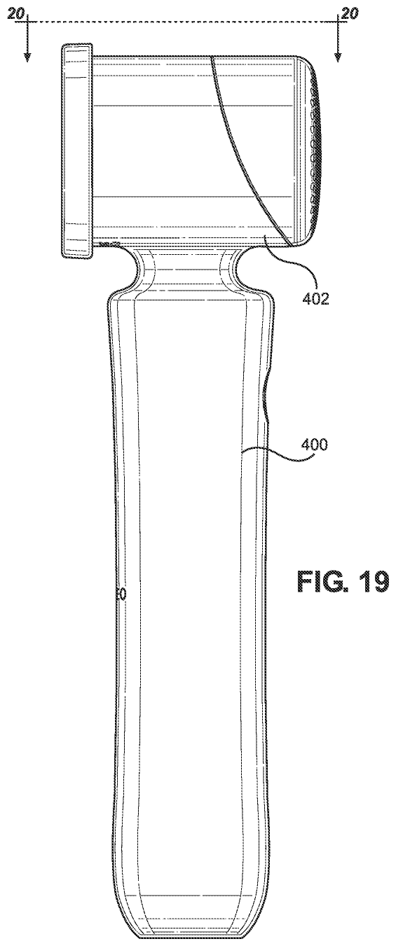

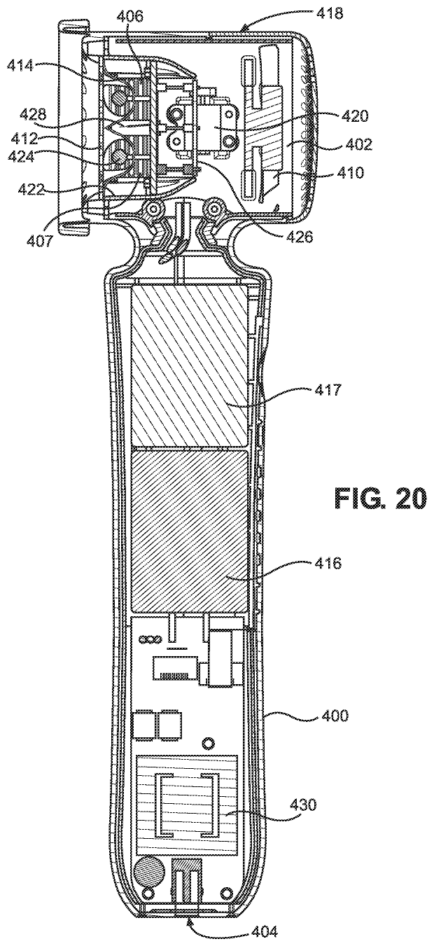

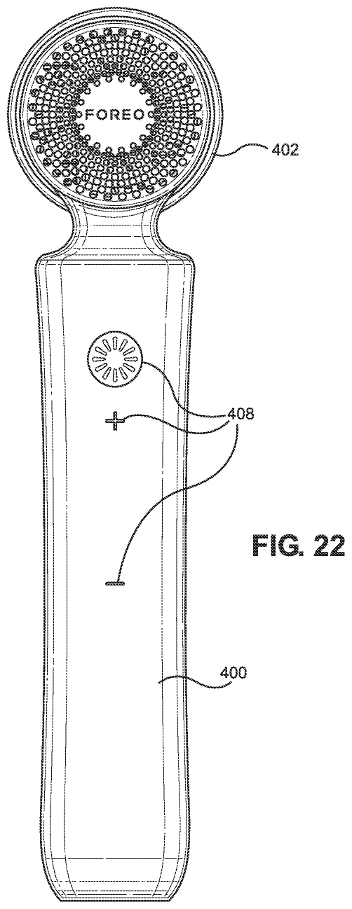

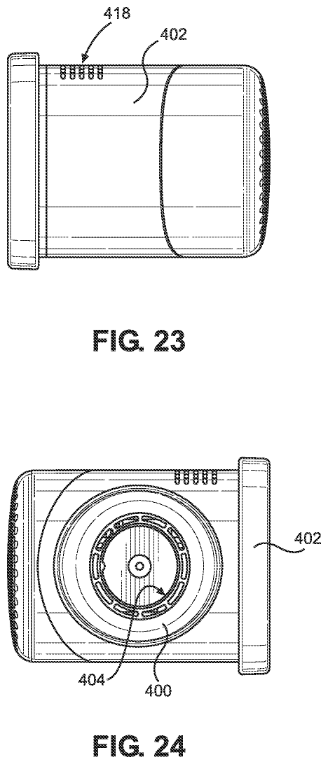

FIGS. 17, 18, 19, 20, 21, 22, 23, and 24 illustrate another example treatment device 6. The treatment device 6 is similar to the treatment device 2 illustrated in FIGS. 1 through 9, except as described below. Thus, the device 6 comprises at least a main body 400, a head 402, a DC jack 404, a first light source 406, a second light source 407, a set of user controls 408, a fan 410, a filter 412, a reflector 414, first and second sensors 416, 417 comprising capacitive sensors, an outlet 418 through which warm air generated by the device 6 can exit the head 402, a motor 420, a bracket 422 configured to contact and hold in place various other components, first and second controllers 424, 426 (which, in some embodiments, contain an interface as described above), a touch sensor 428 disposed in the head 402, a power source 430, and various other internal circuitry and components.

Treatment device 6, however, does not provide a user with EMS treatment, and thus does not include the pins, circuitry, user controls, or other items associated with the same.

The device 6, moreover, includes a set of LED lights 434 which form a circle on the front 432 of the head 402. The LED lights 434 are configured to, at the user's option, emit light while the device is in use. The set of LED lights 434 comprise 18 lights in the illustrated embodiment, which all are circular diameters. The set of LED lights may emit one or more of: green, blue, red, orange, yellow, purple, white, or any other colored light. A skilled artisan will be able to determine how to arrange, number, and configure said set of LED lights in other embodiments based on various considerations, including the size and shape of the head and what type of LED light may be emitted. In other embodiments, no LED light may be emitted from the head and the lights may be removed. In different embodiments, any number of LED lights may be included on the device, but may be arranged in different configurations or may be on other portions of the device, including but not limited to the main body.

Moreover, in this particular embodiment, various components housed within the main body 400 of the device 6 are arranged differently than in prior embodiments described above. A skilled artisan will be able to determine how to arrange, add to, and/or exclude various components described above based on various considerations, including the desired functionality and size of the device.

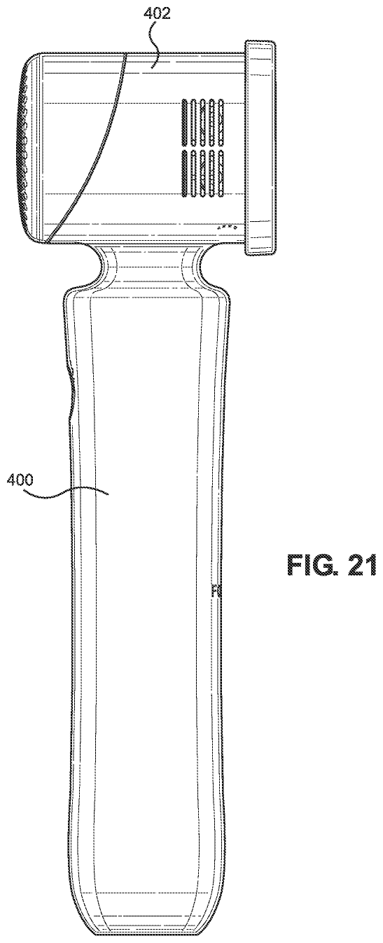

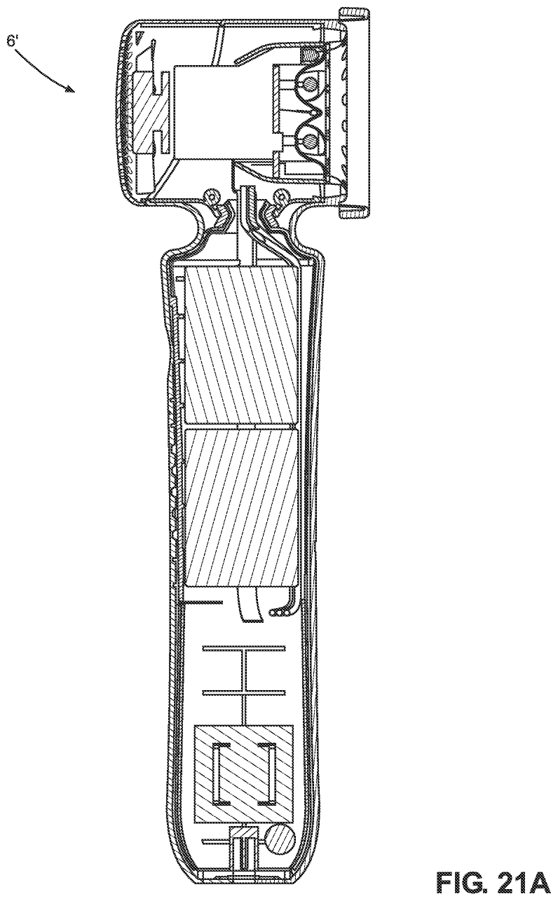

FIG. 21A illustrates an alternative treatment device 6′. Treatment device 6′ includes all components of treatment device 6; however, they are arranged in a slightly different manner.

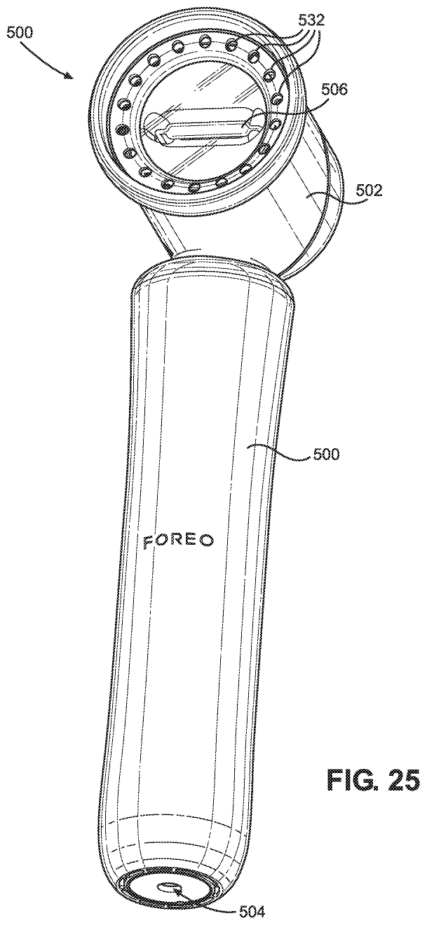

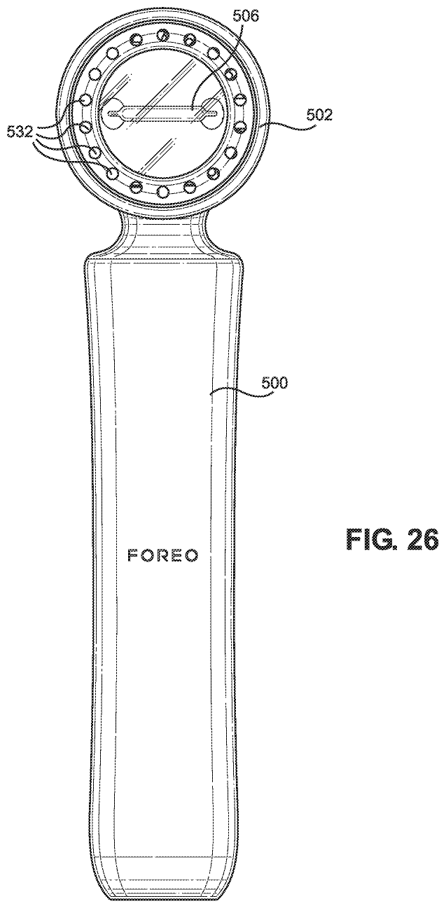

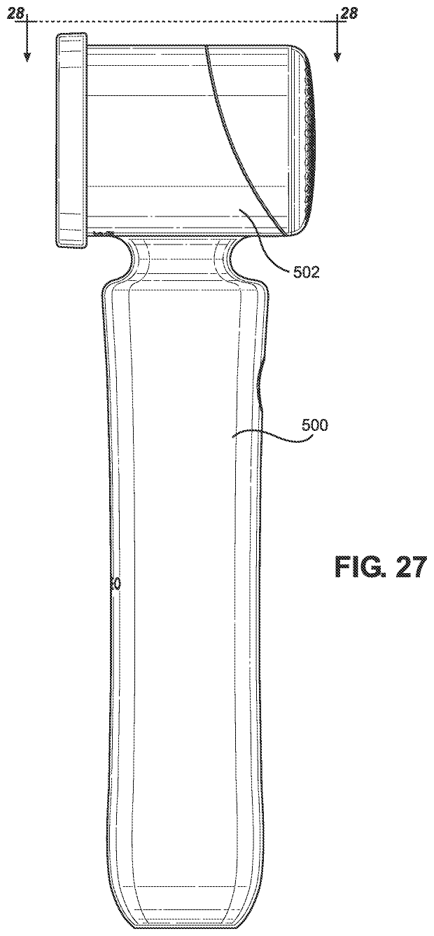

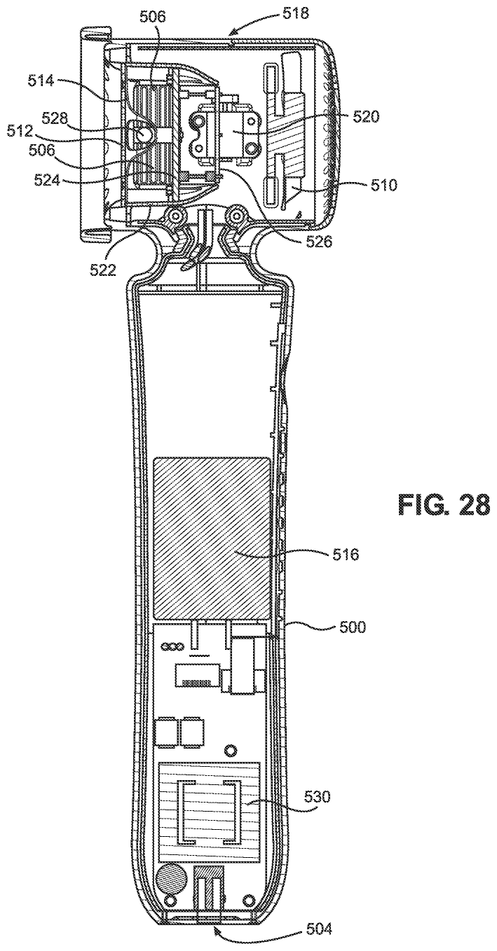

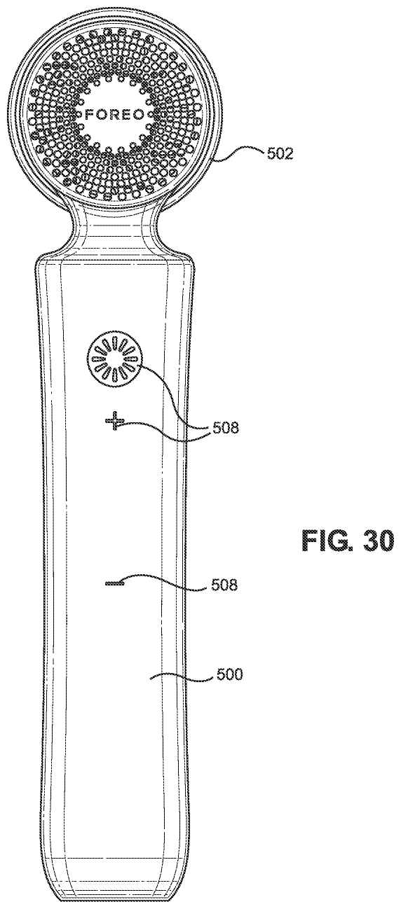

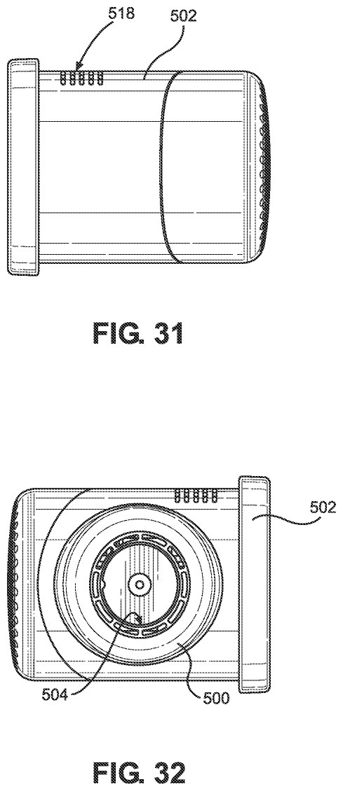

FIGS. 25, 26, 27, 28, 29, 30, 31, and 32 illustrate another example treatment device 8. The treatment device 8 is similar to the treatment device 6 illustrated in FIGS. 17, 18, 19, 20, 21, 22, 23, and 24, except as described below. Thus, the device 8 comprises at least a main body 500, a head 502, a DC jack 504, a first light source 506, a set of user controls 508, a fan 510, a filter 512, a reflector 514, a first sensor 516 comprising a capacitive sensor, an outlet 518 through which warm air generated by the device 8 can exit the head 502, a motor 520, a bracket 522 configured to contact and hold in place various components, first and second controllers 524, 526 (which, in some embodiments, contain an interface as described above), a touch sensor 528 disposed in the head 502, a power source 530, a set of LED lights 532, and various other internal circuitry and components.

Treatment device 8 does not provide a user with EMS treatment (which is similar to treatment device 6), and thus does not include the pins, circuitry, user controls, or other items associated with the same. Additionally, in this embodiment the treatment device 8 does not include a second light source or a second sensor, contrasting the device 8 with that of treatment device 6.

Moreover, in this particular embodiment, various components housed within the main body 500 of the device 8 are arranged differently than in prior embodiments described above. A skilled artisan will be able to determine how to arrange, add to, and/or exclude various components described above based on various considerations, including the desired functionality and size of the device.

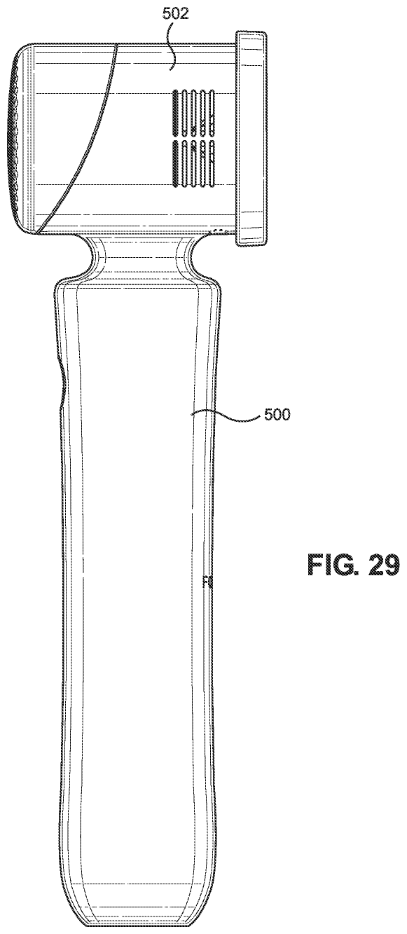

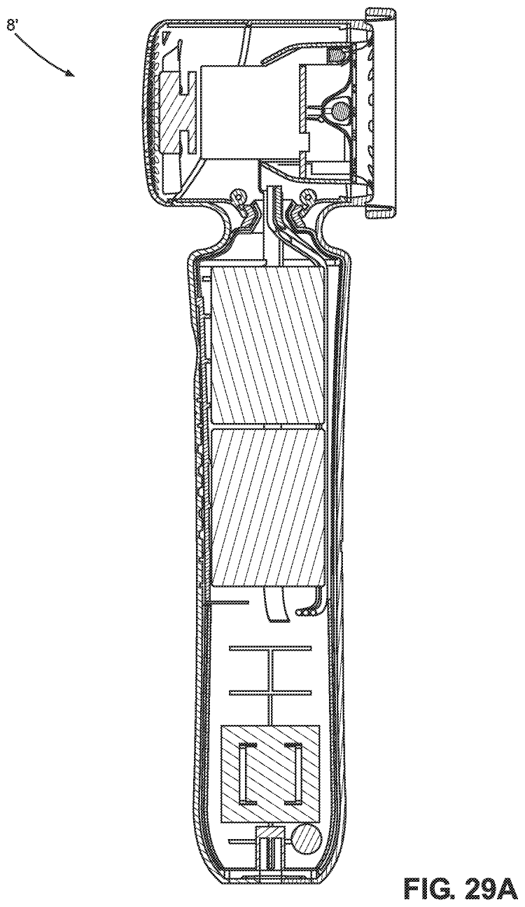

FIG. 29A illustrates an alternative treatment device 8′. Treatment device 8′ includes all components of treatment device 8; however, they are arranged in a slightly different manner.

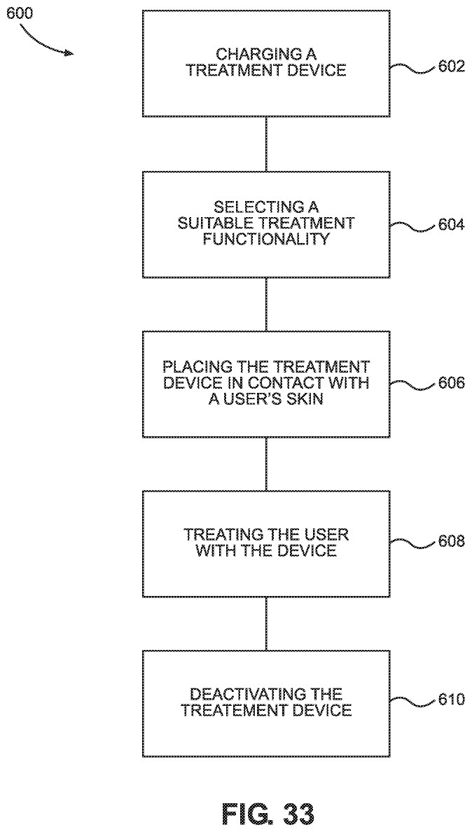

FIG. 33 is a flowchart representation of an example method 600 of using a treatment device. Performance of this method results in the treatment of the user's body, such as the face and/or neck, by a treatment device. This method can be used for treating any portion of a human body.

An initial step 602 comprises charging a treatment device, such as treatment device 2 (or one of treatment devices 4, 6, or 8), by connecting the device to a power source via a DC jack.

Another step 604 comprises selecting a suitable treatment functionality, such as IPL treatment and/or vibration stimulation, or any combination of suitable treatments. A treatment pattern may be selected via the user control. Selection of a particular treatment pattern may be communicated to a user via informational lights, which may emit different patterns, colors, or flashes of light to indicate the various treatment patterns.

Another step 606 comprises placing the treatment device in contact with or adjacent the user. Doing so allows the treatment device to treat the skin via the selected treatment functionality.

Another step 608 comprises treating the user with the device.

Another step 610 comprises deactivating the treatment device. Deactivation of the treatment device may occur automatically at the end of a pre-set treatment or via manipulation of the user control.

It is noted that it is advantageous to complete this method 600 in the order illustrated and described. However, any order is considered suitable.

In all examples, a treatment device and its various components may be formed of any suitable material, including presently known and later-developed materials. A skilled artisan will be able to select appropriate materials for an example treatment device based on various considerations, including the size and shape of the treatment device, the motor housed within the treatment device, the light source housed within the treatment device, and the particular treatments desired.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A treatment device, comprising:
a main body having a first end, a second end substantially opposite the first end, an intermediate portion extending from the first end to the second end, a head disposed adjacent the first end;
a first light source disposed within the head and configured to emit light adjacent the head;
a second light source disposed within the head and configured to emit light adjacent the head;
a motor disposed within the main body, the motor configured to produce pulsations of said treatment device;
a capacitor disposed within the main body, the capacitor configured to supply power to said device; and
a set of user controls disposed on the intermediate portion, the set of user controls configured to operate the first and second light sources, and the motor.

2. The treatment device of claim 1, wherein the first light source comprises a discharge xenon lamp.

3. The treatment device of claim 2, wherein the second light source comprises a discharge xenon lamp.

4. The treatment device of claim 3, the first light source and second light source are substantially parallel to one another on separate planes.

5. The treatment device of claim 4, wherein the main body includes a silicone exterior covering the intermediate portion.

6. The treatment device of claim 5, wherein the first light source emits light at a wavelength between about 400 nanometers and about 800 nanometers.

7. The treatment device of claim 6, wherein the first light source emits light at a wavelength between about 500 nanometers and 650 nanometers.

8. The treatment device of claim 7, wherein the second light source emits light at a wavelength between about 400 nanometers and about 800 nanometers.

9. The treatment device of claim 8, wherein the second light source emits light at a wavelength between about 500 nanometers and 650 nanometers.

10. The treatment device of claim 9, wherein the head is at least partially comprised of metal.

11. The treatment device of claim 10, wherein the head comprises a series of passageways extending from a first exterior surface of the head to a first interior surface of the head.

12. A treatment device, comprising:
a main body having a first end, a second end substantially opposite the first end, an intermediate portion extending from the first end to the second end, a head disposed adjacent the first end;
a first light source disposed within the head and configured to emit light adjacent the head;
a first capacitor disposed within the main body, the capacitor configured to supply power to the first light source;
a second capacitor disposed within the main body, the capacitor configured to supply power to the second light source; and
a set of user controls disposed on the intermediate portion, the set of user controls configured to operate the first and second light sources and the motor;
wherein the main body includes a silicone exterior covering the intermediate portion.

13. The treatment device of claim 12, wherein each of the first and second light sources comprise discharge xenon lamps.

14. The treatment device of claim 13, wherein each of the first and second light sources emit light at a wavelength between about 500 nanometers and 650 nanometers.

15. The treatment device of claim 14, wherein the device will not provide intense pulsed light stimulation unless said user is in contact with the head of the device.

16. The treatment device of claim 12, wherein the first light source emits light at a wavelength between about 400 nanometers and about 800 nanometers.

17. The treatment device of claim 16, wherein the second light source emits light at a wavelength between about 400 nanometers and 800 nanometers.

18. A treatment device, comprising:
a main body having a first end, a second end substantially opposite the first end, an intermediate portion extending from the first end to the second end, a head disposed adjacent the first end, and a silicone exterior covering at least a portion of the first end, the second end, and the intermediate portion;
a first light source disposed within the head and configured to emit light adjacent the head;
a second light source disposed within the head and configured to emit light adjacent the head;
a motor disposed within the main body, the motor configured to produce pulsations of said treatment device;
a first capacitor disposed within the main body, the capacitor configured to supply power to the first light source;
a second capacitor disposed within the main body, the capacitor configured to supply power to the second light source; and
a set of user controls disposed on the intermediate portion, the set of user controls configured to operate the first and second light sources and the motor.

19. The treatment device of claim 18, wherein the first light source emits light at a wavelength between about 400 nanometers and about 800 nanometers.

20. The treatment device of claim 19, wherein the second light source emits light at a wavelength between about 400 nanometers and 800 nanometers.
